(19) United States
(12) Patent Application Publication
(10) Pub. No.: US 2008/0146846 A1
(43) Pub. Date: Jun. 19, 2008

(54) Direct Amination of Hydrocarbons

(75) Inventors: Harald Dialer, Munchen (DE); Matthias Frauenkron, Freinsheim (DE); Holger Evers, Manncheim (DE); Ekkehard Schwab, Neustadt (DE); Johann-Peter Melder, Bohl-Iggelheim (DE); Frank Rosowski, Mannheim (DE); Frederik Van Laar, Limburgerhof (DE); Joachim-Thierry Anders, Gonnheim (DE); Sven Crone, Limburgerhof (DE); Wolfgang Mackenroth, Bad Durkheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/722,774

(22) PCT Filed: Dec. 20, 2005
(86) PCT No.: PCT/EP05/13699

§ 371 Date: Jun. 25, 2007

(30) Foreign Application Priority Data
Dec. 23, 2004 (DE) 10 2004 062 253.1

(51) Int. Cl. C07C 209/02

(52) U.S. Cl. 564/408

(57) ABSTRACT

The invention relates to a process for preparing nitrogen-containing catalysts, comprising:
a) preparation of an oxidic species comprising the following components:
   at least one metal M selected from groups Ib to VIIb and VIII of the Periodic Table of the Elements, it being possible for the same metal to be present in different oxidation states;
   if appropriate one or more promoters P selected from groups Ib to VIIb and VIII of the Periodic Table of the Elements, the lanthanides, and from groups IIIa to VIa of the Periodic Table of the Elements, excluding oxygen and sulfur;
   if appropriate one or more elements R selected from hydrogen, alkali metals and alkaline earth metals;
   if appropriate one or more elements Q selected from chloride and sulfate;
   oxygen, the molar proportion of oxygen being determined by the valency and frequency of the elements in the oxidic species other than other oxygen;
b) reaction of the oxidic species with an amine component selected from ammonia, primary and secondary amines and ammonium salts,
   the nitrogen-containing catalyst being formed with the formation of water, and to nitrogen-containing catalysts preparable by this process. The invention further relates to a process for aminating hydrocarbons using the inventive nitrogen-containing catalyst and to the use of an oxidic species in a process for the direct amination of hydrocarbons.

The invention relates to a process for the direct amination of hydrocarbons, to catalysts which are used in the direct amination and to a process for preparing these catalysts.

The commercial preparation of amines, in particular of aromatic amines such as aniline, is typically carried out in multistage reactions. Aniline is prepared, for example, typically by converting benzene to a benzene derivative, e.g. nitrobenzene, chlorobenzene or phenol, and subsequent conversion of this derivative to aniline.

More advantageous than such indirect processes for preparing amines, in particular aromatic amines, are methods which enable a direct preparation of the amines from the corresponding hydrocarbons. Numerous processes for the direct amination of hydrocarbons, in particular aromatic hydrocarbons, e.g. benzene, are known, in which oxidic catalysts are used.

CA 553,988 discloses a process for preparing aniline from benzene, in which benzene, ammonia and gaseous oxygen are reacted over a platinum catalyst at a temperature of about 1000° C. Suitable platinum-containing catalysts are platinum alone, platinum with certain specific metals and platinum together with certain specific metal oxides. In addition. CA 553.988 discloses a process for preparing aniline, in which benzene in the gas phase is reacted with ammonia in the presence of a reducible metal oxide at temperatures of from 100 to 1000° C. without addition of gaseous oxygen. Suitable reducible metal oxides are the oxides of iron, nickel, cobalt, tin, antimony, bismuth and copper.

U.S. Pat. No. 3,919,155 relates to the direct amination of aromatic hydrocarbons with ammonia, in which the catalyst used is nickel/nickel oxide, and the catalyst may additionally comprise oxides and carbonates of zirconium, strontium, barium, calcium, magnesium, zinc, iron, titanium, aluminum, silicon, cerium, thorium, uranium and alkali metals.

U.S. Pat. No. 3,929,889 likewise relates to the direct amination of aromatic hydrocarbons with ammonia over a nickel/nickel oxide catalyst, the catalysts used having been partly reduced to elemental nickel and subsequently reoxidized to obtain a catalyst which has a ratio of nickel:nickel oxide of from 0.001:1 to 10:1.

U.S. Pat. No. 4,001,260 relates to a process for the direct amination of aromatic hydrocarbons with ammonia, in which a nickel/nickel oxide catalyst is used, and is applied to zirconium dioxide and has been reduced with ammonia before use in the amination reaction.

U.S. Pat. No. 4,031,106 relates again to the direct amination of aromatic hydrocarbons with ammonia over a nickel/nickel oxide catalyst on a zirconium dioxide support which further comprises an oxide selected from lanthanoids and rare earth metals.

WO 00/09473 relates to a process for preparing amines by direct amination of aromatic hydrocarbons over a catalyst comprising at least one vanadium oxide.

WO 99/10311 relates to a process for the direct amination of aromatic hydrocarbons at a temperature of <500° C. and a pressure of <10 bar. The catalyst used is a catalyst comprising at least one metal selected from transition metals, lanthanides and actinides, preferably Cu, Pt, V, Rh and Pd. Preference is given to carrying out the direct amination in the presence of an oxidizing agent to increase the selectivity and/or the conversion.

WO 00/69804 relates to a process for the direct amination of aromatic hydrocarbons, in which the catalyst used is a complex comprising a noble metal and a reducible metal oxide. Particular preference is given to catalysts comprising palladium and nickel oxide or palladium and cobalt oxide.

All of the processes mentioned start from a mechanism for direct amination as detailed in the abstract of WO 00/69804. According to this, the desired amine compound is initially prepared under noble metal catalysis from the aromatic hydrocarbon and ammonia, and the hydrogen formed in the first step is “scavenged” in a second step with a reducible metal oxide. The same mechanistic considerations form the basis of the process in WO 00/09473, in which the hydrogen is scavenged with oxygen from vanadium oxides (page 1, lines 30 to 33). The same mechanism also forms the basis in U.S. Pat. No. 4,001,260, as is evident from the remarks and the diagram in column 2, lines 16 to 44.

It is an object of the present invention to provide catalysts in whose presence the direct amination of hydrocarbons proceeds with outstanding selectivity and in comparatively good yields under conditions which can be performed on the industrial scale, and a process for preparing these catalysts and a process for direct amination in which these catalysts are used.

This object is achieved by a process for preparing nitrogen-containing catalysts, comprising:
a) preparation of an oxidic species comprising the following components:
   at least one metal M selected from groups Ib to VIIb and VIII of the Periodic Table of the Elements (CAS version), it being possible for the same metal to be present in different oxidation states;
   if appropriate one or more, preferably from 0 to 3, promoters P, for example P¹, P² and P³, selected from groups Ib to VIIb and VIII of the Periodic Table of the Elements, the lanthanides, and from groups IIIa to VIa of the Periodic Table of the Elements, excluding oxygen and sulfur;
   if appropriate one or more elements R selected from hydrogen, alkali metals and alkaline earth metals;
   if appropriate one or more elements Q selected from chloride and sulfate;
   oxygen, the molar proportion of oxygen being determined by the valency and frequency of the elements in the oxidic species other than other oxygen;
b) reaction of the oxidic species with an amine component selected from ammonia, primary and secondary amines and ammonium salts,

the nitrogen-containing catalyst being formed with the formation of water.

The nitrogen-containing catalysts preparable by the process according to the invention are highly active in the direct amination of hydrocarbons. The preparation of the nitrogen-containing catalysts makes it possible to undertake an exact adjustment of the required amount of amine component and thus to enable an optimal composition of the starting substances in order to achieve optimal yields and selectivities. Such an optimal adjustment of the starting substances has not been possible to date, since, as already stated, there is no formation of nitrogen-containing catalysts, as claimed in the present application, in the processes of the prior art.

In the process of the present application, it is possible that steps a) and b) are effected simultaneously, i.e. the amine component is added actually during the preparation of the oxidic species. However, it is also possible to carry out steps a) and b) successively, by first forming the oxidic species and then reacting it with the amine component, preference being given to the latter.

Metals M used with preference are metals of group Ib, VIIb and VIII of the Periodic Table of the Elements (CAS version). Particularly preference is given to using the following metals or metal combinations: Ni, Co, Mn, Fe, Ru, Ag and/or Cu. The metals M used may each be present in various oxidation states.

The metals M used are even more preferably Ni and/or Co, which may be present in various oxidation states.

Especially preferably, the metal M used is nickel which may be present in various oxidation states in the nitrogen-containing catalyst.

In addition, the oxidic species may comprise one or more, preferably from 0 to 3, more preferably from 1 to 3, promoters P, for example P¹, P² and P³, selected from groups Ib to VIIb and VIII of the Periodic Table of the Elements (CAS version), the lanthanides, and groups IIIa and IVa of the Periodic Table of the Elements (CAS version). The promoter or the promoters is/are more preferably selected from boron, aluminum, and also silicon and germanium, the lanthanides, in particular cerium, praseodymium, neodymium, promethium, samarium, europium, gadolinium, and groups Ib and IIIb to VIb, VIIb and VIII of the Periodic Table of the Elements (CAS version), preferably groups Ib, IIIb, IVb, VIb, VIIb and VIII, in particular copper, manganese, cobalt, lanthanum, titanium, zirconium, hafnium, Mg, Al, rhodium, rhenium, ruthenium, palladium, platinum, silver, molybdenum and tungsten.

Very particular preference is given to using at least one promoter P selected from copper, manganese, cobalt, rhodium, rhenium, ruthenium, palladium, platinum, silver, zirconium, molybdenum and tungsten. The promoter P may, if appropriate, be present in the form of its oxide and/or oxide hydroxide.

The metals used as metal M or as promoters P may be present in the form of alloys In this case, the metals used as metals M or as promoters P may each form alloys with one another, or at least one metal M may form alloys with at least one promoter P. Examples of alloys are alloys of nickel and cobalt, or alloys of copper and nickel, and these alloys may additionally be alloyed with at least one metal selected from the group consisting of Rh, Re, Ru, Pd, Pt and Ag. In addition, alloys of nickel and at least one metal of the aforementioned group are conceivable.

In the context of the present application, alloys are understood to be both alloys of different metals and alloys of different metal oxides or alloys of one or more metals with one or more metal oxides.

It is known to those skilled in the art that some of the above-listed metals M or P are generally not present in pure form, but rather together with a further “related” metal which is generally to be found in the same group of the Periodic Table of the Elements. For example, zirconium is present together with hafnium, and cerium together with lanthanum and/or neodymium. In the context of the present application, for example, zirconium and cerium should thus not be understood only to be the pure metals, but rather may comprise small amounts, known to those skilled in the art, of related metals. In this case, the aforementioned metals may also be present in the form of their metal oxides.

Furthermore, the oxidic species may comprise one or more elements R selected from alkali metals, in particular lithium, sodium and potassium, alkaline earth metals, in particular magnesium, calcium, strontium and barium.

In addition, the oxidic species may comprise one or more elements Q selected from chloride and sulfate.

Finally, the oxidic species comprises oxygen, the molar proportion of the oxygen being determined by the valency and frequency of the elements in the oxidic species other than oxygen.

In a preferred embodiment of the process according to the invention, the oxidic species comprises the following components
at least one metal M selected from group VIII of the Periodic Table of the Elements, preferred metals already having been listed above, it being possible for the same metal to be present in different oxidation states;
at least one promoter P selected from groups Ib to VIIb and VIII of the Periodic Table of the Elements (CAS version), the lanthanides, and groups IIIa and IVa of the Periodic Table of the Elements (CAS version), preferred embodiments of the promoter already having been listed above, and
oxygen, the molar proportion of the oxygen being determined by the valency and frequency of the elements in the oxidic species other than oxygen.

In a particularly preferred embodiment of the process according to the invention, the oxidic species comprises the following components:
nickel and/or cobalt, preferably nickel, as the metal M, it being possible for nickel and/or cobalt to present in different in oxidation states,
at least one promoter P selected from the group consisting of Cu, Co, Mo, W and Mn, preferably Cu, Mo and W, preference being given to using either Cu alone as a promoter P¹ or Cu together with Mo and, if appropriate, W, particular preference being given to the latter, it being possible for the at least one promoter P¹ to be present at least partly in the form of its oxides, and Cu is preferably present in the form of an alloy with nickel,
if appropriate, at least one further promoter P³ selected from the group consisting of Rh, Re, Ru, Pd, Pt and Ag, preferably Rh or Ag, it being possible for the at least one further promoter P³ to be present at least partly in the form of an alloy with nickel and or copper;
a support material in the form of inorganic oxides selected from the group consisting of ZrO₂, SiO₂, Al₂O₃, MgO, TiO₂, B₂O₃, CaO, ZnO, BaO, ThO₂, CeO₂, Y₂O₃ and mixtures of these oxides, for example magnesium aluminum oxide, preferably TiO₂, ZrO₂, Al₂O₃, magnesium aluminum oxide and SiO₂, more preferably ZrO₂ and magnesium aluminum oxide.
The aforementioned oxides may be present at least partly in the form of oxide hydroxides. In the context of the present application, the aforementioned oxides are thus to be understood not only to be the oxides but also oxide hydroxides or mixtures of oxides and oxide hydroxides.

The magnesium aluminum oxide support material, which is used with particular preference in addition to ZrO₂, may be prepared by any processes known to those skilled in the art. Preference is given to using magnesium aluminum oxide which is obtainable by calcination of hydrotalcite or hydrotalcite-like compounds. A suitable process for preparing magnesium aluminum oxide, comprising the step of calcining hydrotalcite or hydrotalcite-like compounds, is disclosed, for example, in Catal. Today 1991. 11, 173 or in “Comprehensive Supramolecular Chemistry”, (Eds. Albertl, Bein), Pergamon, N.Y., 1996, Vol. 7, 251.

The oxidic species as per the aforementioned particularly preferred embodiment may be used directly as a catalyst system in a process for the direct amination of hydrocarbons with amines. Suitable hydrocarbons and amines are mentioned below, the suitable amines corresponding to the amine component mentioned below. The process conditions for the direct amination of hydrocarbons are known to those skilled in the art,

In general, the direct amination is effected at temperatures of from 200 to 600° C., preferably from 200 to 500° C., more preferably from 300 to 400° C. The reaction pressure in the amination, preferably in the amination of benzene, is generally from 1 to 900 bar, preferably from 1 to 500 bar, more preferably from 1 to 300 bar. In a further preferred embodiment of the amination process according to the invention, the reaction pressure is less than 30 bar, preferably from 1 to <25 bar, more preferably from 3 to 10 bar. Suitable hydrocarbons are the hydrocarbons which are mentioned above.

The present application further provides for the use of the oxidic species as defined in the aforementioned embodiments in a process for the direct amination of hydrocarbons. When it is used as a catalyst system in a process for the direct amination of hydrocarbons, the desired aminated hydrocarbon is obtained with high selectivity at good conversions of the hydrocarbon used. Suitable process conditions and reactants are specified below.

The oxidic species which is used in accordance with the invention and is suitable as a catalyst system in the direct amination thus most preferably comprises, in addition to nickel and/or cobalt, preferably nickel, ZrO₂, or magnesium aluminum oxide as a support material, and also Cu as a promoter P¹ and molybdenum, tungsten and/or manganese, preferably molybdenum and/or tungsten, as further promoters P¹ and, if appropriate, a promoter P³, preferably Rh or Ag. Nickel and/or cobalt and Cu may be present fully or partly in the form of their oxides

Very particular preference is given to the use of an oxidic species consisting of from 80% by weight, preferably from 20 to 65% by weight, of nickel and/or cobalt and copper, preferably nickel and copper, from 0.1 to 10% by weight, preferably from 0.5 to 5% by weight, of molybdenum, tungsten and/or manganese, preferably molybdenum and/or tungsten, from 5 to 60% by weight, preferably from 10 to 25% by weight, of Zr, it being possible for Zr to be present in the form of ZrO₂, and also oxygen, the molar proportion of oxygen being determined by the valency and amount of the non-oxygen elements nickel and/or cobalt, Cu, Mo, W, Mn and Zr, the sum total of the components in the oxidic species being 100% by weight. Furthermore, very particular preference is given to the use of an oxidic species consisting of the aforementioned components, the oxidic species having. Instead of from 5 to 60% by weight, preferably from 10 to 25% by weight, of Zr, Zr being present in the form of ZrO₂, from 5 to 60% by weight, preferably from 10 to 25% by weight, of Mg+Al, Mg+Al being in the form of magnesium aluminum oxide, and, instead of from 0.1 to 10% by weight, preferably from 0.5 to 5% by weight, of molybdenum, tungsten and/or manganese, preferably molybdenum and/or tungsten, from 0 to 10% by weight, preferably from 0 to 5% by weight, of molybdenum, tungsten and/or manganese, preferably molybdenum and/or tungsten.

A further particularly preferred embodiment relates to the use of an oxidic species consisting of the aforementioned components, which comprises either Zr in the form of ZrO₂ or Mg+Al in the form of magnesium aluminum oxide, the oxidic species comprising at least partly instead of copper.

In a further very particularly preferred embodiment, the present application relates to the use of an oxidic species consisting of from 10 to 80% by weight, preferably from 20 to 65% by weight, of nickel and/or cobalt and copper, preferably nickel and copper, from 0.1 to 10% by weight, preferably from 0.5 to 5% by weight, of molybdenum, tungsten and/or manganese, preferably molybdenum and/or tungsten, from 0.1 to 5% by weight, preferably from 0.5 to 2% by weight, of Rh or Ag, from 5 to 60% by weight, preferably from 10 to 25% by weight, of Zr, Zr being present in the form of ZrO₂, and oxygen, the molar proportion of oxygen being determined by the valency and amount of the non-oxygen elements nickel and/or cobalt, Cu, Mo, W, Mn, Rh or Ag and Zr, the sum total of the components in the oxidic species being 100% by weight. Furthermore, very particular preference is given to the use of an oxidic species consisting of the aforementioned components, the oxidic species having, instead of from 5 to 60% by weight, preferably from 10 to 25% by weight, teing present in the form of ZrO₂, from 5 to 60% by weight, preferably from 10 to 25% by weight, of Mg+Al, Mg+Al being present in the form of magnesium aluminum oxide, and, instead of from 0.1 to 10% by weight, preferably from 0.5 to 5% by weight, of molybdenum, tungsten and/or manganese, preferably molybdenum and/or tungsten, from 0 to 10% by weight, preferably from 0 to 5% by weight, of molybdenum, tungsten and/or manganese, preferably molybdenum and/or tungsten.

In the aforementioned particularly preferred embodiments of the oxidic species, copper and nickel or copper, nickel and cobalt may be present at least partly in the form of alloys. These alloys may additionally be alloyed with Rh or Ag. In this context, alloys are understood to be alloys of the metals mentioned and alloys of the oxides of the metals mentioned and alloys of one or more metals and one or more metal oxides.

Nickel and/or cobalt and copper are present in the oxidic species preferably in at least two different oxidation states, in the form of nickel and nickel oxide or cobalt and cobalt oxide and copper and copper oxide. The molar nickel/nickel oxide ratio or molar cobalt/cobalt oxide ratio and the molar copper/copper oxide ratio are more preferably from 0 to 500, even more preferably from 0.0001 to 50 and in particular from 0.005 to 5. The copper oxide may either be copper(I) oxide or copper(II) oxide, or a mixture of copper(l) oxide and copper(II) oxide. In the preferred oxidic species, in a further embodiment. Cu may be replaced at least partly by Ag. Ag may occur in the form of Ag(I) oxide, AgNO₃ or in metallic form or alloyed with M-MOₓ where M is a suitable metal and MOₓ suitable metal oxide. In this context suitable metals or metal oxides are understood to be metals or metal oxides which are present in the oxidic species and can be alloyed with Ag.

In a preferred embodiment of the process for preparing the nitrogen-containing catalysts, the oxidic species is prepared in step a) by the following steps:
aa) precipitation of the desired metal compounds from a solution of their salts, for example of the nitrates, by addition of the base, for example ammonium carbonate, sodium hydroxide, ammonium hydroxide, lithium hydroxide, sodium carbonate, sodium hydrogencarbonate, potassium carbonate or mixtures thereof, to form the corresponding metal oxides or metal oxide hydroxides;
ab) filtering, washing and drying of the metal oxides or metal oxide hydroxides to obtain oxidic complexes;
ac) if appropriate calcination;
ad) if appropriate reduction of the resulting oxidic complexes with hydrogen; and
ae) if appropriate reoxidation with a defined amount of oxygen in order to obtain the desired oxidic species,

it being possible to carry out either step ac) or steps ad) and ae) or steps ac), ad) and ae).

The reoxidation with a defined amount of oxygen in step ad) passivates the oxidic species in a controlled manner. The defined formation of the oxidic species which is active in the direct amination of the hydrocarbons is thus possible by the establishment of the optimal oxidation state(s) of the metal(s). This enables optimal conditions for the formation of the nitrogen-containing catalysts by reaction with the amine component in step b) in the process according to the invention.

The steps ad) (reduction) and ae) (reoxidation) may be dispensed with in the process according to the invention when step ac) (calcination) is carried out.

Steps aa) and ab) detail a preferred embodiment for the preparation of oxidic complexes. It is also possible to obtain the oxidic complexes by impregnation, sol-gel processes, processes with application of freeze-drying, spray-drying and/or suspension and subsequent solvent removal. Also conceivable is a combination of the process preferred according to the present application, comprising steps aa) and ab) (precipitation process), with one of the aforementioned processes.

In the cases in which nitrates are used in step aa) the calcination in step ac) is preferably effected. In general, the calcination is effected at temperatures of from 200 to 800° C., preferably from 300 to 500° C., more preferably from 400 to 500° C. The period of the calcination is generally from 0.25 to 10 h, preferably from 0.5 to 7.5 h, more preferably from 1.5 to 5 h.

The reduction of the resulting oxidic complexes with hydrogen in step ad) is effected with the aid of hydrogen at temperatures of generally from 100 to 500° C. preferably from 100 to 400° C. more preferably from 150 to 350° C. The pressure is generally from 0.1 to 30 bar, preferably from 0.1 to 20 bar, more preferably from 0.1 to 5 bar.

In step ae) which follows, the reoxidation is effected with a defined amount of oxygen, as already mentioned. This reoxidation is effected generally at temperatures of from 0° C. to 400° C., preferably from 10 to 200° C., more preferably from 20 to 100° C., by, in a preferred embodiment, oxidizing the product from step ad) in a gas stream with an oxygen content rising with time up to a degree of oxidation which is given by the valency and frequency of the elements other than oxygen.

In a preferred embodiment of the process according to the invention, the metal M is cobalt and/or nickel, preferably nickel, and at least one promoter P¹ is Cu, which are present in at least two oxidation states, and the reoxidation in step ad) is effected with an amount of oxygen which is required to attain a molar metal/metal oxide ratio of from 0 to 500, preferably from 0.0001 to 50, more preferably from 0.005 to 5. It is likewise possible to carry out the direct amination on the basis of the fully oxidized metals nickel and/or cobalt and copper in the oxidic species when NH₃ is used as the amine component in the direct amination. In a further embodiment, the process according to the invention may be carried out with an oxidic species in which Cu is replaced at least partly by Ag.

In step b) of the process according to the invention, the oxidic species is reacted with an amine component selected from ammonia, primary and secondary amines and ammonium salts. This forms the desired nitrogen-containing catalyst with formation of water. Preference is given to using amine components which are suitable for introducing a —NRR′ unit in the hydrocarbon used, where R and R′ are each independently H, alkyl or aryl, preferably H, methyl or ethyl, more preferably H. Amine components used with preference are ammonia, ammonium salts, for example ammonium chloride, ammonium nitrate, ammonium carbonate and ammonium carbamate, substituted amines, for example alkylamines such as methylamine or other primary alkylamines, hydroxylamines, alkoxy amines or hydrazines. In addition, the amine component may be a compound which forms ammonia in situ when it is decomposed under the reaction conditions in the process of the present application (for example urea). The amine components used are more preferably ammonia, primary alkylamines and ammonium salts such as ammonium chloride, ammonium nitrate, ammonium carbonate or ammonium carbamate.

When a gaseous amine component is used in the process according to the invention, for example ammonia or methylamine, the reaction of the oxidic species is effected in step b) generally at temperatures of from −35 to 600° C., preferably from 25 to 450° C., more preferably from 50 to 400° C. The pressure is generally from 0.1 to 350 bar, preferably from 1 to 50 bar, more preferably from 1 to 20 bar. The reaction with the amine * component is generally carried out for a period for from 0.001 to 10 hours preferably from 0.01 to 5 hours, more preferably from 0.1 to 1 hour.

When the oxidic species is reacted in step b) of the process according to the invention with a liquid or solid amine component (for example an ammonium salt), the amine component is preferably kneaded into the oxidic species and the nitrogen-containing catalyst is formed by subsequent heating to a temperature of generally from 50 to 600° C., preferably from 50 to 500° C., more preferably from 50 to 400° C. The heating is carried out for a period of generally from 0.1 to 20 hours, preferably from 1 to 15 hours, more preferably from 1 to 10 hours.

Such a reaction of the oxidic species with the amine component results in an intimate mixture between the oxidic species and the amine component. The amine component is thus an integral part of the nitrogen-containing catalyst.

It is assumed that the nitrogen-containing catalyst has the general empirical formula (I)

[MₐP¹_{b}P²_{c}P³_{d}RₑQ_{f}][O]_{g}[NHᵢ]ₕ·j H₂O (I)

where the symbols M, P, for example P¹, P² and P³, R, Q, have already been defined above.

a is from 1 to 100, preferably from 1 to 80, more preferably from 2 to 50;

b is from 0 to 100, preferably from 1 to 80, more preferably from 1 to 50;

c is from 0 to 10, preferably from 1 to 8, more preferably from 2 to 5;

d is from 0 to 10, preferably from 0.01 to 5, more preferably from 0.05 to 2;

e is from 0 to 100, preferably from 1 to 80, more preferably from 2 to 50;

f is from 0 to 100, preferably from 0 to 80, more preferably from 0.1 to 10;

g is from 1 to 250, preferably from 1 to 200, more preferably from 2 to 100:

h is from 1 to 220, preferably from 1.05 to 173, more preferably from 2.0 to 107 (sum of a+b+c+d);

i is from 0 to 3, preferably from 0 to 2;

j is from 0 to 500, preferably from 0 to 100, more preferably from 1 to 80.

The molar ratio between the oxidic species and the amine component, expressed as the ratio

h/(g+h),

in the process according to the invention is generally from 0.0001 to 1, preferably from 0.002 to 0.8, more preferably from 0.01 to 0.6. The addition of a defined amount of the

amine component makes it possible to prepare defined nitrogen-containing catalysts, with the aid of which a direct amination of hydrocarbons, in particular aromatic hydrocarbons, with high selectivity and good yield is possible.

Without being bound to this, the nitrogen-containing catalyst is formed from the oxidic species according to the following equation (using the example of ammonia as the amine component and i=1):

[MₐP¹_{b}P²_{c}P³_{d}RₑQ_{f}][O]_{g+h}·j h₂O+h NH₃→[MₐP¹_{b}P²_{c}P³_{d}RₑQ_{f}][O]_{g}[NH]ₕ·j H₂O+h H₂O

where the symbols M, P, for example P¹, P² and P³, R, Q, a, b, c, d, e, f, g, h, j have already been defined above

The present application further provides nitrogen-containing catalysts preparable by the process according to the invention.

The precise composition of these catalysts is to date unknown. The nitrogen content in the inventive catalysts is generally from 0,0001 to 20% by weight, preferably from 0.1 to 15% by weight, more preferably from 0.1 to 10% by weight. The nitrogen content in the inventive catalysts was determined by means of elemental analysis (combustion in combination with thermoluminescence).

As the metal M, the inventive nitrogen-containing catalyst preferably comprises Ni and/or Co, more preferably Ni. In addition, the inventive catalyst comprises at least one promoter P¹ selected from the group consisting of Cu, Mn, Mo, W and Co, As promoter P¹, the inventive nitrogen-containing catalyst preferably comprises either Cu alone or Cu in combination with Mo and, if appropriate, W. In a further embodiment, the inventive nitrogen-containing catalyst comprises Ag at least partly instead of Cu (alone or in combination with Mo and, if appropriate, W). Furthermore, the catalyst may comprise at least one further promoter P³ selected from the group consisting of Rh, Re, Ru, Mn, Pd, Pt, Ag and Co, preferably Rh and Ag. In the case that Cu is replaced at least partly by Ag, the promoter P³ is not Ag. If appropriate, the catalyst may furthermore comprise a support component selected from CeO₂, Y₂O₃, TiO₂, ZrO₂, Al₂O₃, MgO, magnesium aluminum oxide and SiO₂, preferably ZrO₂ and magnesium aluminum oxide, i.e. the inventive catalyst comprises, if appropriate, at least one promoter P² selected from Ti, Zr, Al, Mg and Si, preferably Zr and (Mg+Al). The inventive nitrogen-containing catalyst thus more preferably comprises Ni and Cu; Ni, Cu and Mo and, if appropriate, W; Ni and Mn; Ni and Ag; Ni, Ag and Mo and, if appropriate W; Ni, Cu and Ag; Ni, Cu, Ag and Mo and, if appropriate, W or Ni and Co, even more preferably Ni and Cu or Ni, Cu and Mo and, if appropriate, W or Ni and Ag or Ni, Ag and Mo and, if appropriate, W or Ni, Cu and Ag or Ni, Cu, Ag and Mo, if appropriate W. Furthermore, the inventive nitrogen-containing catalyst comprises, if appropriate, at least one further promoter P³ and/or at least one further promoter P².

Very particular preference is given to a nitrogen-containing catalyst comprising:
from 10 to 80% by weight, preferably from 25 to 65% by weight, more preferably from 30 to 60% by weight, of at least one metal M selected from Ni and Co, preferably Ni, and Cu as promoter P¹, it being possible for M and Cu to be present at least partly in the form of the corresponding oxides;
from 0 to 50% by weight, preferably from 5 to 40% by weight, more preferably from 10 to 30% by weight, even more preferably from 0.1 to 10% by weight, especially preferably from 0.5 to 5% by weight, of at least one promoter P¹ selected from the group of Mo, W, Mn and Co, preferably Mo, W and Mn, more preferably Mo and W;
from 0 to 60% by weight, preferably from 5 to 60% by weight, more preferably from 10 to 25% by weight, of at least one metal as a promoter P² selected from the group of Ce, Y, Ti, Zr, Ai, Mg and Si, the metal being present in the form of CeO₂, Y₂O₃, TiO₂, ZrO₂, Al₂O₃, magnesium aluminum oxide or SiOz, preferably Zr or (Al+Mg), which is present in the form of ZrO₂ or magnesium aluminum oxide;
from 0 to 10% by weight, preferably from 0.1 to 5% by weight, more preferably from 0.5to 2% by weight, of at least one promoter P³ selected from the group of Rh, Re, Ru, Mn, Pd, Pt and Ag, preferably Rh and Ag;
from 0 to 15% by weight, preferably from 0.1 to 10% by weight, more preferably from 0.5 to 5% by weight, of one or more elements R selected from hydrogen, alkali metals and alkaline earth metals;
from 0 to 5% by weight, preferably from 0 to 2.5% by weight, more preferably from 0.01 to 1% by weight, of one or more elements Q selected from chloride and sulfate; and
oxygen, the molar proportion of oxygen being determined by the valency and frequency of the elements M, P¹, P², P³, R and Q non-oxygen;

where the sum total of the aforementioned components is 100% by weight; and
from 0.0001 to 20% by weight, preferably from 0.1 to 15% by weight, more preferably from 0.1 to 10% by weight, based on the sum total of the aforementioned components, of nitrogen.

In a further preferred embodiment, the present application relates to a nitrogen-containing catalyst which comprises the aforementioned components in the aforementioned amounts, Cu being replaced partly or fully by Ag, and Ag not being comprised additionally as a promoter P³. In the case that Cu is replaced partly or fully by Ag, particular preference is given to no promoter P³ being comprised in the nitrogen-containing catalyst.

Very particular preference is given to a catalyst system consisting of from 10 to 80% by weight, preferably from 20 to 65% by weight, more preferably from 30 to 60% by weight, of nickel and/or cobalt and copper, preferably nickel and copper, from 0.1 to 10% by weight, preferably from 0.5 to 5% by weight, of molybdenum, tungsten and/or manganese, preferably molybdenum and/or tungsten, from 5 to 60% by weight, preferably from 10 to 25% by weight, of Zr, Zr being present in the form of ZrO₂, and oxygen, the molar proportion of oxygen being determined by the valency and amount of the non-oxygen elements nickel and/or cobalt, Cu, Mo, W, Mn and Zr, the sum total of the components in the catalyst system being 100% by weight, and also from 0.1 to 10% by weight, based on the sum total of the aforementioned components, of nitrogen. Furthermore, very particular preference is given to a catalyst system consisting of the aforementioned components, the oxidic species having, instead of from 5 to 60% by weight, preferably from 10 to 25% by weight, of Zr, Zr being present in the form of ZrO₂, from 5 to 60% by weight, preferably from 10 to 25% by weight, of Mg+Al, Mg+Al being present in the form of magnesium aluminum oxide, and, instead of from 0.1 to 10% by weight, preferably from 0.5 to 5% by weight, of molybdenum, tungsten and/or manganese, preferably molybdenum and/or tungsten, from 0 to 10% by weight, preferably from 0 to 5% by weight, of molybdenum, tungsten and/or manganese, preferably molybdenum and/or tungsten.

A further particularly preferred embodiment relates to a catalyst system consisting of the aforementioned components which comprises either Zr in the form of ZrO₂ or Mg+Al in the form of magnesium aluminum oxide, the oxidic species comprising silver instead of copper.

In a further preferred embodiment, the inventive catalyst system consists of from 10 to 80% by weight, preferably from 20 to 65% by weight, more preferably from 30 to 60% by weight, of nickel and/or cobalt and copper, preferably nickel and copper, from 0.1 to 10% by weight, preferably from 0.5 to 5% by weight, of molybdenum, tungsten and/or manganese, preferably molybdenum and/or tungsten, from 0.1 to 5% by weight, preferably from 0.5 to 2% by weignt, of Rh or Ag, from 5 to 60% by weight, preferably from 10 to 25% by weight, of Zr, Zr being present in the form of ZrO₂, and oxygen, the molar proportion of oxygen being determined by the valency and amount of the non-oxygen elements nickel and/or cobalt, Cu, Mo, W, Mn, Rh and Zr, trie sum total of the components in the catalyst system being 100% by weight, and also from 0.1 to 10% by weight, based on the sum total of the aforementioned components, of nitrogen.

Furthermore, very particular presence is given to a catalyst system consisting of the aforementioned components, the catalyst system having, instead of from 5 to 60% by weight, preferably from 10 to 25% by weight, of Zr, Zr being present in the form of ZrO₂, from 5 to 60% by weight, preferably from 10 to 25% by weight, of Mg+Al, Mg+Al being present in the form of magnesium aluminum oxide, and, instead of from 0.1 to 10% by weight, preferably from 0.5 to 5% by weight, of molybdenum, tungsten and/or manganese, preferably molybdenum and/or tungsten, from 0 to 10% by weight, preferably from 0 to 5% by weight, of molybdenum, tungsten and/or manganese, preferably molybdenum and/or tungsten.

Nickel and/or cobalt and copper are present in the oxidic species preferably in at least two different oxidation states in The form of nickel and nickel oxide or cobalt and cobalt oxide or copper and copper oxide. The molar nickel/nickel oxide ratio or molar cobalt cobalt oxide ratio and the molar copper/copper oxide ratio are more preferably from 0 to 500, even more preferably from 0.0001 to 50 and in particular from 0.005 to 5. The copper oxide may either be copper(I) oxide or be copper(II) oxide or mixtures of copper(I) oxide and copper(II) oxide.

Especially preferably, the inventive nitrogen-containing catalyst comprises the elements M, P¹, if appropriate P² and if appropriate P³ in the following combinations:

| | M | P¹ | P² | P³ |
|---|---|---|---|---|
| 1 | Ni | Cu | — | — |
| 2 | Ni | Cu | Zr or (Mg + Al) | — |
| 3 | Ni | Cu | Zr or (Mg + Al) | Rh |
| 4 | Ni | Cu | Zr or (Mg + Al) | Re |
| 5 | Ni | Cu | Zr or (Mg + Al) | Mn |
| 6 | Ni | Cu | Zr or (Mg + Al) | Pd |
| 7 | Ni | Cu | Zr or (Mg + Al) | Pt |
| 8 | Ni | Cu | Zr or (Mg + Al) | Ag |
| 9 | Ni | Cu | Zr or (Mg + Al) | Co |
| 10 | Ni | Cu | Zr or (Mg + Al) | Ru |
| 11 | Ni | Cu, Mo, if | — | — |
| | | appropriate W | | |
| 12 | Ni | Cu, Mo, if | Zr or (Mg + Al) | — |
| | | appropriate W | | |
| 13 | Ni | Cu, Mo, if | Zr or (Mg + Al) | Rh |
| | | appropriate W | | |
| 14 | Ni | Cu, Mo, if | Zr or (Mg + Al) | Re |
| | | appropriate W | | |
| 15 | Ni | Cu, Mo, if | Zr or (Mg + Al) | Mn |
| | | appropriate W | | |
| 16 | Ni | Cu, Mo, if | Zr or (Mg + Al) | Pd |
| | | appropriate W | | |
| 17 | Ni | Cu, Mo, if | Zr or (Mg + Al) | Pt |
| | | appropriate W | | |
| 18 | Ni | Cu, Mo, if | Zr or (Mg + Al) | Ag |
| | | appropriate W | | |
| 19 | Ni | Cu, Mo, if | Zr or (Mg + Al) | Co |
| | | appropriate W | | |
| 20 | Ni | Cu, Mo, if | Zr or (Mg + Al) | Ru |
| | | appropriate W | | |
| 21 | Ni | — | — | — |
| 22 | Ni | Co | — | — |
| 23 | Ni | Co | Zr or (Mg + Al) | — |
| 24 | Ni | Co | Zr or (Mg + Al) | Rh |
| 25 | Ni | Co | Zr or (Mg + Al) | Re |
| 26 | Ni | Co | Zr or (Mg + Al) | Mn |
| 27 | Ni | Co | Zr or (Mg + Al) | Pd |
| 28 | Ni | Co | Zr or (Mg + Al) | Pt |
| 29 | Ni | Co | Zr or (Mg + Al) | Ag |
| 30 | Ni | Co | Zr or (Mg + Al) | Ru |
| 31 | Ni | Mn | — | — |
| 32 | Ni | Mn | Zr or (Mg + Al) | — |
| 33 | Ni | Mn | Zr or (Mg + Al) | Rh |
| 34 | Ni | Mn | Zr or (Mg + Al) | Re |
| 35 | Ni | Mn | Zr or (Mg + Al) | Mn |
| 36 | Ni | Mn | Zr or (Mg + Al) | Pd |
| 37 | Ni | Mn | Zr or (Mg + Al) | Pt |
| 38 | Ni | Mn | Zr or (Mg + Al) | Ag |
| 39 | Ni | Mn | Zr or (Mg + Al) | Co |
| 40 | Ni | Mn | Zr or (Mg + Al) | Ru |
| 41 | Co | Cu | — | — |
| 42 | Co | Cu | Zr or (Mg + Al) | — |
| 43 | Co | Cu | Zr or (Mg + Al) | Rh |
| 44 | Co | Cu | Zr or (Mg + Al) | Re |
| 45 | Co | Cu | Zr or (Mg + Al) | Mn |
| 46 | Co | Cu | Zr or (Mg + Al) | Pd |
| 47 | Co | Cu | Zr or (Mg + Al) | Pt |
| 48 | Co | Cu | Zr or (Mg + Al) | Ag |
| 49 | Co | Cu | Zr or (Mg + Al) | Ru |
| 50 | Co | — | — | — |
| 51 | Co | Mn | — | — |
| 52 | Co | Mn | Zr or (Mg + Al) | — |
| 53 | Co | Mn | Zr or (Mg + Al) | Rh |
| 54 | Co | Mn | Zr or (Mg + Al) | Re |
| 55 | Co | Mn | Zr or (Mg + Al) | Mn |
| 56 | Co | Mn | Zr or (Mg + Al) | Pd |
| 57 | Co | Mn | Zr or (Mg + Al) | Pt |
| 58 | Co | Mn | Zr or (Mg + Al) | Ag |
| 59 | Co | Cu, Mo, if | — | — |
| | | appropriate W | | |
| 60 | Co | Cu, Mo, if | Zr or (Mg + Al) | — |
| | | appropriate W | | |
| 61 | Co | Cu, Mo, if | Zr or (Mg + Al) | Rh |
| | | appropriate W | | |
| 62 | Co | Cu, Mo, if | Zr or (Mg + Al) | Re |
| | | appropriate W | | |
| 63 | Co | Cu, Mo, if | Zr or (Mg + Al) | Mn |
| | | appropriate W | | |
| 64 | Co | Cu, Mo, if | Zr or (Mg + Al) | Pd |
| | | appropriate W | | |
| 65 | Co | Cu, Mo, if | Zr or (Mg + Al) | Pt |
| | | appropriate W | | |
| 66 | Co | Cu, Mo, if | Zr or (Mg + Al) | Ag |
| | | appropriate W | | |
| 67 | Co | Cu, Mo, if | Zr or (Mg + Al) | Ru |
| | | appropriate W | | |

The amounts of the individual components M, P¹, P² and P³ correspond preferably to the amounts specified in the above embodiment, where P¹ in Example 21 and 50 or P² in Examples 1, 11, 21, 22, 31, 41, 50, 51 and 59 or P³ in Examples 1, 2, 11, 12, 21, 22, 23, 31, 32, 41, 42, 50, 51, 52, 59 and 60 are 0, and the sum of the remaining components is 100% by weight.

(Mg+Al) as a promoter P² is understood to be a promoter P² which is present as a carrier material in the form of magnesium aluminum oxide. The magnesium aluminum oxide may be prepared by processes known to those skilled in the art. Preference is given to using magnesium aluminum oxide which is obtainable by calcinations of hydrotalcite or hydrotalcite-like compounds. A suitable process for preparing the magnesium aluminum oxides used with preference is disclosed, for example, in Catal. Today 1991, 11, 73 or in “Comprehensive Supramolecular Chemistry”, (Eds. Alberti, Bein), Pergamon, N.Y., 1996, Vol 7, 251. Very particular preference is given to preparing the magnesium aluminum oxide (the MgAlOx phase) by a coprecipitation of the corresponding metal salts from a supersaturated solution.

The inventive nitrogen-containing catalyst preferably comprises the following combinations of M, P¹, P² and, if appropriate, P³ specified in the table above: 2 to 10, 12 to 20, 42 to 49 or 60 to 67, more preferably 2, 3, 8, 12, 13, 18, 42, 43, 60 or 61, most preferably 2, 3, 8, 12, 13 or 18.

The inventive catalyst is notable for outstanding regeneratability without substantial loss of activity, even after several regeneration cycles. Furthermore, the inventive catalyst may be used in a process for aminating hydrocarbons, the desired aminated hydrocarbon being formed with high selectivity at good conversions of the hydrocarbon used.

The present application thus further provides a process for aminating hydrocarbons, in which the hydrocarbon is contacted with an inventive nitrogen-containing catalyst.

In a preferred embodiment of the amination process according to the invention, the process comprises the following steps:
a) preparation of an oxidic species comprising the following components:
   at least one metal M selected from groups Ib to VIIb and VIII of the Periodic Table of the Elements, it being possible for the same metal to be present in different oxidation states,
   if appropriate one or more, preferably from 0 to 3, promoters P, for example P¹, P² and P³, selected from groups Ib to VIIb and VIII of the Periodic Table of the Elements, the lanthanides, and from groups IIIa to VIa of the Periodic Table of the Elements, excluding oxygen and sulfur;
   if appropriate one or more elements R selected from hydrogen, alkali metals and alkaline earth metals;
   if appropriate one or more elements Q selected from chloride and sulfate;
   oxygen, the molar proportion of oxygen being determined by the valency

and frequency of the elements in the oxidic species other than other oxygen;
b) reaction of the oxidic species with an amine component selected from ammonia, primary and secondary amines and ammonium salts; and
c) addition of the hydrocarbon to be aminated,

where steps b) and c) may be carried out simultaneously, offset in time or successively. Preferred embodiments of the component used in step a) and step b) and preferred reaction conditions of steps a) and b) have already been described above. Steps b) and c) are more preferably effected offset in time. “Offset in time” is understood to mean that the addition of the amine component (step b)) is begun after step a) and, before step b) has ended, the hydrocarbon to be aminated is added (step c)). After step a), the oxidic species formed in step a) is thus initially pretreated with the amine component (step b)). This pretreatment is generally carried out for a period of from 1 to 60 minutes, preferably from 5 to 15 minutes. This forms the inventive nitrogen-containing catalyst Subsequently, while the amine component is still being added, the hydrocarbon to be aminated is added (step c)). Steps b) and c) are effected under the reaction conditions specified below.

However, it is likewise possible that the reaction of the oxidic species with an amine component (step b)) and the addition of the hydrocarbon (step c)) in the amination process according to the invention are effected successfully or simultaneously. In this case too, the inventive nitrogen-containing catalyst which brings about the amination of the hydrocarbon in high selectivities and with good yields is initially formed in situ.

It is possible with the amination process according to the invention to aminate any hydrocarbons, such as aromatic hydrocarbons, aliphatic hydrocarbons and cycloaliphatic hydrocarbons, which may have any substitution and may have heteroatoms and double or triple bonds within their chain or their ring/their rings. In the amination process according to the invention, preference is given to using aromatic hydrocarbons and heteroaromatic hydrocarbons. The corresponding products are the corresponding arylamines or heteroarylamines.

In the context of the present invention, an aromatic hydrocarbon is understood to be an unsaturated cyclic hydrocarbon which has one or more rings and contains exclusively aromatic C—H bonds. The aromatic hydrocarbon preferably has one or more 5- or 6-membered rings.

A heteroaromatic hydrocarbon is understood to be those aromatic hydrocarbons in which one or more of the carbon atoms of the aromatic ring is/are replaced by a heteroatom selected from N, O and S.

The aromatic hydrocarbons or the heteroaromatic hydrocarbons may be substituted or unsubstituted. A substituted aromatic or heteroaromatic hydrocarbon is understood to be a compound in which one or more hydrogen atoms which is/are bonded to a carbon atom or heteroatom of the aromatic ring is/are replaced by another radical. Such radicals are, for example, substituted or unsubstituted alkyl, alkenyl, alkynyl, heteroalkyl, heteroalkenyl, heteroalkynyl, cycloalkyl and/or cycloalkynyl radicals. In addition, the following radicals are possible; halogen, hydroxyl, alkoxy, aryloxy, amino, amido, thio and phosphino. Preferred radicals of the aromatic or heteroaromatic hydrocarbons are selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido, where C₁₋₆ relates to the number of carbon atoms in the main, chain of the alkyl radical, of the alkenyl radical or of the alkynyl radical, and C₃₋₈ to the number of carbon atoms of the cycloalkyl or cycloalkenyl ring. It is also possible that the substituents (radicals) of the substituted aromatic or heteroaromatic hydrocarbon have further substituents.

The number of substituents (radicals) of the aromatic or heteroaromatic hydrocarbon is arbitrary. In a preferred embodiment, the aromatic or heteroaromatic hydrocarbon has, however, at least one hydrogen atom which is bonded directly to a carbon atom or a heteroatom of the aromatic ring. Thus, a 6-membered ring preferably has 5 or fewer substituents (radicals) and a 5-membered ring preferably has 4 or fewer substituents (radicals). A 6-membered aromatic or heteroaromatic ring more preferably has 4 or fewer substituents, even more preferably 3 or fewer substituents (radicals). A 5-membered aromatic or heteroaromatic ring preferably bears 3 or fewer radicals, more preferably 2 or fewer radicals.

In a particularly preferred embodiment of the process according to the invention, an aromatic or heteroaromatic hydrocarbon of the general formula

(A)-(B)ₙ

is used, where the symbols are each defined as follows:
A is independently aryl or heteroaryl, A is preferably selected from phenyl, diphenyl, benzyl, dibenzyl, naphthyl, anthracene, pyridyl and quinoline:

n is from 0 to 5, preferably from 0 to 4, especially in the case when A is a 6-membered aryl or heteroaryl ring; in the case that A is a 5-membered aryl or heteroaryl ring, n is preferably from 0 to 4; irrespective of the ring size, n is more preferably from 0 to 3, most preferably from 0 to 2 and in particular from 0 to 1; the remaining carbon atoms or heteroatoms of A which do not bear any substituents B bear hydrogen atoms, or, if appropriate, no substituents;
B is independently selected from the group consisting of alkyl, alkenyl, alkynyl, substituted alkyl, substituted alkenyl, substituted alkynyl, heteroalkyl, substituted heteroalkyl, heteroalkenyl, substituted heteroalkenyl, heteroalkynyl, substituted heteroalkynyl, cycloalkyl, cycloalkenyl, substituted cycloalkyl, substituted cycloalkenyl, halogen, hydroxy, alkoxy, aryloxy, carbonyl, amino, amido, thio and phosphino; B is preferably independently selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido.

The term “independently” means that, when n is 2 or greater, the substituents B may be

identical or different radicals from the groups mentioned.

In the present application, alkyl is understood to mean branched or unbranched, saturated acyclic hydrocarbyl radicals. Examples of suitable alkyl radicals are methyl, ethyl, n-propyl, i-propyl, n-butyl, t-butyl, i-butyl, etc. The alkyl radicals used preferably have from 1 to 50 carbon atoms, more preferably from 1 to 20 carbon atoms, even more preferably from 1 to 6 carbon atoms and in particular from 1 to 3 carbon atoms.

In the present application, alkenyl means branched or unbranched, acyclic hydrocarbyl radicals which have at least one carbon-carbon double bond. Suitable alkenyl radicals are, for example, 2-propenyl, vinyl, etc. The alkenyl radicals have preferably from 2 to 50 carbon atoms, more preferably from 2 to 20 carbon atoms, even more preferably from 2 to 6 carbon atoms and in particular from 2 to 3 carbon atoms. The term alkenyl also encompasses radicals which have either a cis-orientation or a trans-orientation (alternatively E or Z orientation).

In the present application, alkynyl is understood to mean branched or unbranched, acyclic hydrocarbyl radicals which have at least one carbon-carbon triple bond. The alkynyl radicals preferably have from 2 to 50 carbon atoms, more preferably from 2 to 20 carbon atoms, even more preferably from 1 to 6 carbon atoms and in particular from 2 to 3 carbon atoms.

Substituted alkyl, substituted alkenyl and substituted alkynyl are understood to mean alkyl, alkenyl and alkynyl radicals in which one or more hydrogen atoms which are bonded to one carbon atom of these radicals are replaced by another group. Examples of such other groups are heteroatoms, halogen, aryl, substituted aryl, cycloalkyl, cycloalkenyl, substituted cycloalkyl, substituted cycloalkenyl and combinations thereof. Examples of suitable substituted alkyl radicals are benzyl, trifluoromethyl, inter alia.

The terms heteroalkyl, heteroalkenyl and heteroalkynyl refer to alkyl, alkenyl and alkynyl radicals in which one or more of the carbon atoms in the carbon chain is replaced by a heteroatom selected from N, O and S. The bond between the heteroatom and a further carbon atom may be saturated, or, if appropriate, unsaturated.

According to the present application, cycloalkyl is understood to mean saturated cyclic nonaromatic hydrocarbyl radicals which are composed of a single ring or a plurality of fused rings. Suitable cycloalkyl radicals are, for example, cyclopentyl, cyclohexyl, cyclooctyl, bicyclooctyl, etc. The cycloalkyl radicals have preferably between 3 and 50 carbon atoms, more preferably between 3 and 20 carbon atoms, even more preferably between 3 and 8 carbon atoms in particular between 3 and 6 carbon atoms.

According to the present application, cycloalkenyl is understood to mean partly unsaturated, cyclic nonaromatic hydrocarbyl radicals which have a single fused ring or a plurality of fused rings. Suitable cycloalkenyl radicals are, for example, cyclopentenyl, cyclohexenyl, cyclooctenyl, etc. The cycloalkenyl radicals have preferably from 3 to 50 carbon atoms, more preferably from 3 to 20 carbon atoms, even more preferably from 3 to 8 carbon atoms and in particular from 3 to 6 carbon atoms.

Substituted cycloalkyl and substituted cycloalkenyl radicals are cycloalkyl and cycloalkenyl radicals, in which one or more hydrogen atoms of any carbon atom of the carbon ring is replaced by another group. Such other groups are, for example, halogen, alkyl, alkenyl, alkynyl substituted alkyl, substituted alkenyl, substituted alkynyl, aryl, substituted aryl, cycloalkyl, cycloalkenyl, substituted cycloalkyl, substituted cycloalkenyl, an aliphatic heterocyclic radical, a substituted aliphatic heterocyclic radical, heteroaryl, substituted heteroaryl, alkoxy, aryloxy, boryl, phosphino, amino, silyl, thio, seleno and combinations thereof. Examples of substituted cycloalkyl and cycloalkenyl radicals are 4-dimethylaminocyclohexyl, 4,5-dibromocyclohept-4-enyl, inter alia.

In the context of the present application, aryl is understood to mean aromatic radicals which have a single aromatic ring or a plurality of aromatic rings which are fused, joined via a covalent bond or joined by a suitable unit, for example; a methylene or ethylene unit. Such suitable units may also be carbonyl units, as in benzophenol, or oxygen units, as in diphenyl ether, or nitrogen units, as in diphenylamine. The aromatic ring or the aromatic rings are, for example, phenyl, naphthyl, diphenyl, diphenyl ether, diphenylamine and benzophenone. The aryl radicals preferably have from 6 to 50 carbon atoms, more preferably from 6 to 20 carbon atoms, most preferably from 6 to 8 carbon atoms.

Substituted aryl radicals are aryl radicals in which one or more hydrogen atoms which are bonded to carbon atoms of the aryl radical are replaced by one or more other groups. Suitable other groups are alkyl, alkenyl, alkynyl, substituted alkyl, substituted alkenyl, substituted alkynyl, cycloalkyl, cycloalkenyl, substituted cycloalkyl, substituted cycloalkenyl, heterocyclo, substituted hetereocyclo, halogen, halogen-substituted alkyl (e.g. CF₃), hydroxyl, amino, phosphino, alkoxy, thio and both saturated and unsaturated cyclic hydrocarbyl radicals which may be fused on the aromatic ring or on the aromatic rings or may be joined by a bond, or may be joined to one another via a suitable group Suitable groups have already been mentioned above.

According to the present application, heterocyclo is understood to mean a saturated, partly unsaturated or unsaturated, cyclic radical in which one or more carbon atoms of the radical are replaced by a heteroatom, for example N, O or S. Examples of heterocyclo radicals are piperazinyl, morpholinyl, tetrahydropyranyl, tetrahydrofuranyl, piperidinyl, pyrrolidinyl, oxazolinyl, pyridyl, pyrazyl, pyridazyl, pyrimidyl.

Substituted heterocyclo radicals are those heterocyclo radicals in which one or more hydrogen atoms which are bonded to one of the ring atoms are replaced by another group. Suitable other groups are halogen, alkyl, substituted alkyl, aryl, substituted aryl. heteroaryl, substituted heteroaryl, alkoxy, aryloxy, boryl, phosphino, amino, silyl, thio, seleno and combinations thereof.

Alkoxy radicals are understood to be radicals of the general formula -OZ¹ in which Z¹ is selected from alkyl, substituted alkyl, cycloalkyl, substituted cycloalkyl, heterocycloalkyl, substituted heterocycloalkyl, silyl and combinations thereof. Suitable alkoxy radicals are, for example, methoxy, ethoxy, benzyloxy, t-butoxy, etc. The term aryloxy is understood to mean those radicals of the general formula -OZ¹ in which Z¹ is selected from aryl, substituted aryl, heteroaryl, substituted heteroaryl and combinations thereof Suitable aryloxy radicals are phenoxy, substituted phenoxy, 2-pyridinoxy, 8-quinolinoxy, inter alia.

Amino radicals are understood to be radicals of the general formula -NZ¹Z² in which Z¹ and Z² are each independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

Aromatic or heteroaromatic hydrocarbons used with preference in the amination process according to the invention are selected from benzene, naphthalene, anthracene, toluene, xylene, phenol and aniline, and also pyridine, pyrazine, pyndazine, pyrimidine and quinoline. It is also possible to use mixtures of the aromatic or heteroaromatic hydrocarbons mentioned. Particular preference is given to using the aromatic hydrocarbons benzene, naphthalene, anthracene, toluene, xylene, phenol and aniline, very particular preference to using benzene, toluene and aniline. Especially preferably, benzene is used in the amination process according to the invention, so that the product formed is aniline.

The reaction conditions in the amination process according to the invention are dependent upon factors including the aromatic hydrocarbon to be aminated and the catalyst used.

The amination, preferably the amination of benzene, which is used with very particular preference as the aromatic hydrocarbon, is effected generally at temperatures of from 200 to 600° C., preferably from 200 to 500° C., more preferably from 250 to 450° C. and most preferably from 300 to 400° C.

The reaction pressure in the amination, preferably in the amination of benzene, is generally from 1 to 900 bar, preferably from 1 to 500 bar. more preferably from 1 to 300 bar. In a preferred embodiment of the amination process according to the invention, the reaction pressure is preferably from 50 to 300 bar, more preferably from 100 to 300 bar, most preferably from 150 to 300 bar. In a further preferred embodiment of the amination process according to the invention, the reaction pressure is less than 30 bar, preferably from 1 to <25 bar, more preferably from 3 to 10 bar. It has been found that, surprisingly, the process according to the invention can be carried out at low pressure with good yield and selectivities, preferably using inventive catalysts comprising preferably Ni and Cu; Ni, Cu, Mo and, if appropriate, W; Ni and Mn or Ni and Co and, if appropriate, at least one further promoter P³ selected from the group of Rh, Re, Ru, Mn, Pd and Ag, preferably Rh and Ag. Particularly preferred catalysts have already been mentioned above. The temperature of the amination process according to the latter embodiment corresponds to the abovementioned temperature.

The resonance time in the amination process according to the invention, preferably in the amination of benzene, is generally from 15 minutes to 8 hours, preferably from 15 minutes to 4 hours, more preferably from 15 minutes to 1 hour, in the case of performance in a batchwise process. In the case of performance in a continuous process, the resonance time is generally from 0.1 second to 20 minutes, preferably 0.5 second to 10 minutes.

The relative amount of the hydrocarbon used and of the amine component is dependent upon the amination reaction carried out and the reaction conditions. In general, at least stoichiometric amounts of the hydrocarbon and the amine component are used. However, it is typically preferred to use one of the reaction partners in a stoichiometric excess in order to achieve a shift in the equilibrium to the side of the desired product and at a higher conversion. Preference is given to using the amine component in a stoichiometric excess.

The amination process according to the invention proceeds with outstanding selectivity. The selectivity is determined by the following equation:

$$\% selectivity = \frac{Mass of the amination product prepared ( x 100 )}{{( Mass of the HC used )}_{at the start} {( Mass of the HC used )}_{end}}$$

$$HC = ( hydrocarbon )$$

In general, it is possible with the process according to the invention in a conversion of benzene to aniline to achieve selectivities of generally at least 90%, preferably of at least 93%, more preferably of at least 95%, even more preferably of at least 97% and in particular of at least 98%.

The conversion of hydrocarbon is calculated according to the present application as follows:

$$\% conversion = \frac{\vert {( Amount of the HC )}_{at the start} - {( Amount of the HC )}_{end} \vert}{{( Amount of the HC )}_{at the start}}$$

$$HC = ( hydrocarbon )$$

The reaction pressure in the amination, preferably in the amination of benzene, is generally from 1 to 900 bar, preferably from 1 to 500 bar, more preferably from 1 to 300 bar. In a preferred embodiment of the amination process according to the invention, the reaction pressure is preferably from 50 to 300 bar, more preferably from 100 to 300 bar, most preferably from 150 to 300 bar. In a further preferred embodiment of the amination process according to the invention, the reaction pressure is less than 30 bar, preferably from 1 to <25 bar. more preferably from 3 to 10 bar. It has been found that, surprisingly, the process according to the invention can be carried out with a good yield and selectivities at low pressure.

For the particularly preferred amination of benzene to aniline at a reaction pressure of preferably from 50 to 300 bar, more preferably from 100 to 300 bar, most preferably from 150 to 300 bar, the conversions are generally at least 5%, preferably at least 10%, more preferably at least 15%, most preferably at least 20%.

For the likewise particularly preferred amination of benzene to aniline at a reaction pressure of less than 30 bar, preferably from 1 to <25 bar, more preferably from 3 to 10 bar, the conversions are generally at least 2%, preferably at least 5%, more preferably at least 10%, even more preferably at least 15%, especially preferably at least 20%.

The amination process according to the invention, using the inventive nitrogen-containing catalysts, is thus notable for outstanding selectivities and very good conversions in comparison to the prior art.

The amination process according to the invention may be carried out continuously, batchwise or semicontinuously. Suitable reactors are thus both stirred tank reactors and tubular reactors. Typically reactors are, for example, high pressure stirred tank reactors, autoclaves, fixed bed reactors, fluidized bed reactors, moving beds, circulating fluidized beds, salt bath reactors, plate heat exchangers as reactors, tray reactors having a plurality of trays with or without heat exchange or drawing/feeding of substreams between the trays, in possible designs as radial flow or axial flow reactors, continuous stirred tanks, steel reactors, etc. and the reactor suitable in each case for the desired reaction conditions (such as temperature, pressure and residence time) is used. The reactors may each be used as a single reactor, as a series of individual reactors and/or in the form of two or more parallel reactors. The reactors may be operated in an AB mode (alternating mode). The process according to the invention may be carried out as a batch reaction, semicontinuous reaction or continuous reaction. The specific reactor construction and performance of the reaction may vary depending on the amination process to be carried out, the state of matter of the aromatic hydrocarbon to be aminated, the required reaction times and the nature of the nitrogen-containing catalyst used. Preference is given to carrying out the process according to the invention for direct amination in a high pressure stirred tank reactor, fixed bed reactor or fluidized bed reactor.

In a particularly preferred embodiment, a fixed bed or fluidized bed reactor is used in the amination of benzene to aniline.

The hydrocarbon and the amine component may be introduced in gaseous or liquid form into the reaction zone of the particular reactor. The preferred phase is dependent in each case upon the amination carried out and the reactor used. In a preferred embodiment, for example in the preparation of aniline from benzene, benzene and ammonia are preferably preset as gaseous reactants in the reaction zone. Typically, benzene is fed as a liquid which is heated and evaporated to form a gas, while ammonia is present either in gaseous form or in a supercritical phase in the reaction zone. It is likewise possible that benzene is present in a supercritical phase.

The hydrocarbon and the amine component may be introduced together into the reaction zone of the reactor, for example as a premixed reactant stream, or separately. In the case of a separate addition, the hydrocarbon and the amine component may be introduced simultaneously, offset in time or successively into the reaction zone of the reactor. Preference is given to adding the amine component and adding the hydrocarbon offset in time. In this case, the oxidic species is pretreated with the amine component and the hydrocarbon is added subsequently, during the further addition of the amine component. A definition of the term “offset in time” is given above. In the case of a simultaneous addition of hydrocarbon and amine component too, the inventive nitrogen-containing catalyst which brings about the amination of the hydrocarbon in high selectivities and with good yields is formed initially.

If appropriate, further coreactants, cocatalysts or further reagents are introduced into the reaction zone of the reactor in the process according to the invention, depending in each case on the amination carried out. For example, in the amination of benzene, oxygen or an oxygen-containing gas may be introduced into the reaction zone of the reactor. The relative amount of gaseous oxygen which can be introduced into the reaction zone is variable and depends upon factors including the catalyst system used. The molar ratio of gaseous oxygen to aniline may, for example, be in the range from 0.05:1 to 1:1, preferably from 0.1:1 to 0.5:1. However, it is also possible to carry out the amination of benzene without addition of oxygen or an oxygen-containing gas into the reaction zone.

After the amination, the desired product is isolated by processes known to those skilled in the art.

In a preferred embodiment of the present application, the catalyst system used is regenerated fully or at least partly after it has been used in the amination reaction. The present application thus further provides a process for aminating hydrocarbons, comprising the steps of:
i) reaction of a hydrocarbon with the inventive nitrogen-containing catalyst to form an at least partly reduced catalyst system which is free of nitrogen or has a reduced nitrogen content compared to the inventive nitrogen-containing catalyst,
ii) at least partial regeneration of the at least partly reduced catalyst system to form an oxidic species which, if appropriate, has a reduced nitrogen content compared to the partly reduced catalyst system: suitable oxidic species already having been mentioned above;
iii) reaction of the oxidic species, if appropriate, has a reduced nitrogen content compared to the partly reduced catalyst system with an amine component selected from ammonia, primary and secondary amines and ammonium salts;

it being possible to steps iii) and i) to be effected simultaneously or offset in time, or step iii) is effected first and then i). Preference is given to effecting steps iii) and i) offset in time, the definition of “offset in time” having been specified above.

Suitable amine components and processes for reacting the oxidic species with the amine component (step iii) have already been mentioned above (see process step b) of the aforementioned process according to the invention). Suitable reaction conditions have likewise been mentioned above (see process step c) of the aforementioned process according to the invention).

In the context of the present application, “at least partly reduced” is understood to mean that a regeneration can be carried out when nickel oxide is still present in the catalyst system, i.e. when not all of the nickel oxide present in the catalyst has been reduced to nickel, or when the promoter P¹ is still present in the form of its oxide and has not yet been reduced fully.

The term “at least partial regeneration” is understood to mean that regeneration in step (ii) does not have to be effected until all of the nickel or the entire amount of the promoter P¹ is present in the same oxidation states in the catalyst system as before the amination was carried out. If appropriate, nickel or the promoter P¹ is oxidized fully. However, preference is given to fully reoxidizing the nickel or the promoter P¹ to the oxidation states which are present in the inventive catalyst system before the amination is carried out, i.e. a full regeneration, it is likewise possible to carry out the direct amination with a fully oxidized catalyst system, in which case a partial reduction can in this case be effected by ammonia as the amine component.

The regeneration (reoxidation) may be effected either in the reaction zone of the reactor or outside the reactor, by subjecting the at least partly reduced catalyst system to oxidizing conditions with reoxidation of the nickel and, if appropriate, of the promoter P¹. Suitable oxidizing conditions are, for example, the treatment of the at least partly reduced catalyst system with an oxygen-comprising gas, for example air, or with oxygen, at a temperature of generally from 200 to 800° C., preferably from 300 to 600° C., more preferably from 300 to 450° C. The duration of the reoxidation is dependent upon the catalyst system and the amount of the metals M and, if appropriate, P¹ to be oxidized. For example, the reoxidation can last from generally 10 minutes to 10 hours, preferably from 30 minutes to 5 hours. In one embodiment, the entire catalyst system disposed in the reaction zone can be regenerated simultaneously without the catalyst system being removed from the reaction zone, by changing the conditions in the reactor from the reaction conditions which are established for an amination reaction to the abovementioned regeneration conditions. This regeneration of the entire catalyst is possible in particular in stirred tank reactors and also continuous reactors with a fixed bed or a fluidized bed. However, it is also possible in principle, for example in fluidized bed reactors, to withdraw a portion of the catalyst system continuously or batchwise from the reaction zone and to regenerate it externally and subsequently to feed it continuously or discontinuously back to the reaction zone.

In one embodiment of the process according to the invention, step i) (reaction of a hydrocarbon with the inventive nitrogen-containing catalyst), ii) (regeneration) and iii) (reaction of the oxidic species with the amine component) are carried out successively, and steps i), ii) and iii) are each passed through repeatedly. There is thus a cyclic procedure (amination—regeneration—formation of the nitrogen-containing catalyst—amination . . . ). In general, steps i), ii) and iii) in the process according to the invention using the inventive nitrogen-containing catalyst may be passed through from two to 10⁷ times, preferably from 10² to 10⁶ times, more preferably from 10³ to to 10⁵ times, without a significant loss of activity of the inventive catalyst occurring. As mentioned above, it is likewise possible that step iii) and step i) are carried out simultaneously and step ii) is carried out after step i). In addition, as likewise mentioned above, steps iii) and i) may be carried out offset in time, which is preferred.

However, it is also possible to carry out the regeneration in step ii) of the process according to the invention in parallel to the reaction of step i) of the process according to the invention.

The present application therefore further provides a process according to the invention comprising steps i), ii) and iii), in which the regeneration in step ii) is carried out in parallel to the reaction in step i). This may be achieved, for example, by admixing oxygen or an oxygen comprising gas, for example air, to the reactants used in a continuous performance of the amination process according to the invention.

In general, a treatment of the catalyst system regenerated as detailed above with hydrogen is not required. The use of catalyst systems without a promoter P³ is thus likewise possible. According to the invention, the present application thus also comprises catalyst systems and oxidic species which do not comprise a promoter P³ or another noble metal. However, such a treatment under reducing conditions can be carried out before the reaction of the oxidic species with the amine component to prepare the inventive nitrogen-containing catalyst.

Without being bound to a theory, it is presumed that the inventive amination of hydrocarbons and subsequent regeneration of the oxidic species proceeds by the following steps (illustrated using the example of ammonia as the amine component and i=1, which is not obligatory):

[MₐP¹_{b}P²_{c}P³_{d}RₑQ_{f}][O]_{g}[NHᵢ]ₕ·jH₂O (nitrogen-containing complex)+k (A)−(B)ₙ, (aromatic hydrocarbon)→[MₐP¹_{b}P²_{c}P³_{d}RₑQ_{f}][O]_{g}[NHᵢ]ₕ₋ₖ]·jH₂O+k H₂N−(A)−(B)ₙ.

or

i) MₐP¹_{b}P²_{c}RₑQ_{f}[O]_{g+h} (oxidic species)·jH₂O+hNH₃→[MₐP¹_{b}P²_{c}P³_{d}RₑQ_{f}][O]_{g}[NHᵢ]ₕ·jH₂O+hH₂O

ii) [MₐP¹_{b}P²_{c}RₑQ_{f}][O]_{g}[NHᵢ]ₕ·jH₂O+k (A)−(B)ₙ→[MₐP¹_{b}P²_{c}P³_{d}RₑQ_{f}[O]_{g}[NHi]ₕ₋ₖ+k H₂N−(A)−(B)ₙ
(where i=1)

In these formulae, k≦h and “h−k” means the residual amount of nitrogen present in the oxidic species after direct amination. Steps i) and ii) may be effected successively or in parallel.

The oxidic species is regenerated with oxygen or an oxygen-containing compound according to the following scheme (illustrated by way of example using an oxidic species, which does not contain a residual amount of [NHᵢ]):

[MₐP¹_{b}P²_{c}P³_{d}RₑQ_{f}][O]_{g}·jH₂O+h ½ O₂→[MₐP₁^{b}P₂^{c}P₃^{d}RₑQ^{f}][O _{g+h}·jH₂O (=oxidic species)

The symbols mentioned have already been explained above.

With the aid of the invention nitrogen-containing catalyst, of the process according to the invention for preparing this catalyst, and the amination process according to the invention, it is possible to prepare a large number of amines starting from hydrocarbons, the amination process according to the invention proceeding with outstanding selectivities and very good yields.

The present application further relates to the use of the inventive nitrogen-containing catalysts in a process for aminating hydrocarbons. Preference is given to carrying out the process for aminating hydrocarbons as has been described above. Preferentially suitable nitrogen-containing catalysts and hydrocarbons have likewise been described above.

The examples which follow illustrate the invention additionally.

### EXAMPLES

### Comparative Example 1

### According to DE-A 39 19 155

System: NiO/Ni/ZrO₂:

2 mol of nickel and 0.6 mol of zirconium are dissolved in the form of their nitrate salts in 6000 ml of water. A solution of 2.8 mol of ammonium carbonate in 3000 ml of water is added dropwise to this solution and the mixture is subsequently stirred at 65° C. overnight. Subsequently, the resulting reaction mixture is filtered and washed with demineralized water. The resulting solid is dried at 110° C. in a drying cabinet for 113 hours. After the drying, the solid is substantially comminuted, calcined under air at 450° C. for 4 hours and reduced. The reduction is carried out 380° C. reduction being effected first with 10% H₂ in N₂ for 10 minutes, then with 25% H₂ in N₂ for 10 minutes, then with 50% H₂ in N₂ for 10 minutes, then with 75% H₂ in N₂ for 10 minutes and finally with 100% H₂ for 3 hours. The % are each % by volume.

This catalyst is used to carry out an amination of benzene with NH₃. For the amination. 16.9 g of the catalyst are initially charged in an autoclave and 20.3 g of NH₃ and 39 g of benzene are added under an initial helium pressure of 40 bar. The reaction is effected at 350° C. and about 300 bar (autogenous pressure). From 2.0 to 3.8% aniline are obtained with a selectivity of from 95 to 98% The variations of selectivity and yield are caused by slightly different heating and cooling times.

### Comparative Example 2

### According to WO 00/69804 and Applied Catalysis A: General 227 (2002) 43)

System: Rh, Ni—Mn Impregnated on K—TiO₂

Nickel nitrate and manganese nitrate (the amount of nickel nitrate and manganese nitrate are calculated from the composition of the resulting catalyst system) are mixed together with a 10% by weight rhodium nitrate solution and heated to 70° C. For complete dissolution, another 2 ml of water are added. A TiO₂ support material which comprises K (K—TiO₂) is impregnated with this solution. After drying at 110: C and calcining at 450° C. for 4 hours, a catalyst system comprising 11.9-12% by weight of Ni, 0.3-1% by weight of Mn and 1.1% by weight of Rh is obtained, and these components together with the support material add up to 100% by weight.

This catalyst is used to carry out an amination of benzene with NH₃. The amination is effected under the reaction conditions specified in Comparative Example 1. From 1.0 to 1.4% aniline is obtained with a selectivity of from 96 to 98%. After reoxidation and reuse under the aforementioned reaction conditions, the yield of aniline falls to from 0.6 to 0.7% and the selectivity to from 60 to 70%,

### Inventive Example 3

System: Ni/NiO—Cu/CuO—MoO₃—ZrO₂

The catalyst is Prepared According to DE-A 44 28 004 (Catalyst A):

An aqueous solution of nickel nitrate, copper nitrate and zirconium acetate, which comprises 4.48% by weight of Ni (calculated as NiO), 1.52% by weight of Cu (calculated as CuO) and 2.28% by weight of Zr (calculated as ZrO₂), is precipitated simultaneously in a stirred vessel in a constant stream with a 20% aqueous sodium carbonate solution at a temperature of 70° C., in such a way that the pH, measured with a glass electrode, of 7.0 is maintained. The resulting suspension is filtered and the filtercake is washed with demineralized water until the electrical conductivity of the filtrate is approx. 20 μS. Sufficient ammonium heptamolybdate is then incorporated into the moist filtercake that the above-specified oxide mixture is obtained Afterward, the filtercake is dried at a temperature of 150° C. in a drying cabinet or a spray drier. The hydroxide-carbonate mixture obtained in this way is then heat-treated at a temperature of from 430 to 460° C. over a period of 4 hours. The thus prepared oxidic species has the composition: 50% by weight of NiO, 17% by weight of CuO, 1.5% by weight of MoO₃ and 31.5% by weight of ZrO₂. The reduction is carried out at 190C. reduction being effected first with 10% H₂ in N₂ for 10 minutes, subsequently with 25% H₂ in N₂ for 10 minutes, then with 50% H₂ in N₂ for 10 minutes, then 75% H₂ in N₂ for 10 minutes and finally with 100% H₂ for 3 hours. The % are each % by volume. The reoxidation of the reduced oxidic species is carried out at room temperature in diluted air (air in N₂ with a maximum O₂ content of 5% by volume).

This catalyst is used to carry out amination of benzene with NH₃. The amination is effected under the reaction conditions specified in Comparative Example 1 in an autoclave at 350° C. and 300 bar. From 4.5 to 6% aniline are obtained with a selectivity of 98%.

### Inventive Example 4

The catalyst system according to Example 3 is tested in Example 4 at a pressure of 9 bar and a temperature of 350° C. in a continuous method: To this end, 320 g of the oxidic species are initially converted by reaction with ammonia (18 mol/h) to the inventive nitrogen-containing catalyst (T=350° C., p=9 bar). Subsequently, the nitrogen-containing catalyst system is reacted with benzene (2 mol/h) at a pressure of 9 bar. Space-time yields (STY) of from 20 to 25 g/kg_{cat,h} are achieved, and the selectivity is from 98 to 99.5%. The catalyst system may be regenerated oxidatively and, after conversion to a nitrogen-containing catalyst system, reused in the direct amination.

1-15. (canceled)

16. A process for aminating aromatic hydrocarbons selected from benzene, naphthalene, anthracene, toluene, xylene, phenol, aniline, pyridine, pyrazine, pyridazine, pyrimidine and quinoline, which comprises contacting the hydrocarbon with the nitrogen-containing catalyst preparable by a process comprising:
a) preparation of an oxidic species comprising the following components:
nickel as metal M, it being possible for the nickel to be present in different oxidation states;
Cu together with Mo as promotors P¹ and optionally at least one further promotor P³, selected from the group consisting of Rh, Re, Ru, Pd, Pt and Ag, wherein the at least one further promotor P may—at least partly—be alloyed with nickel and/or copper; and a support material in form of ZrO₂;
if appropriate one or more elements R selected from hydrogen, alkali metals and alkaline earth metals;
if appropriate one or more elements Q selected from chloride and sulfate;
oxygen, the molar proportion of oxygen being determined by the valency and frequency of the elements in the oxidic species other than other oxygen;
b) reaction of the oxidic species with an amine component selected from ammonia, primary and secondary amines and ammonium salts,
the nitrogen-containing catalyst being formed with the formation of water; or with an oxidic species preparable by step a) of the process for preparing the nitrogen-containing catalyst.

17. The process according to claim 16, comprising the following steps:
preparation of a nitrogen-containing catalyst by the process according to claim 16, comprising steps a) and b) and
c) addition of the hydrocarbon to be aminated,
it being possible for the oxidic species to be reacted with an amine component according to step b) and the hydrocarbon to be added (step c)) simultaneously, offset in time or successively.

18. The process for animating hydrocarbons according to claim 16, comprising the steps of:
i) reaction of a hydrocarbon with the nitrogen-containing catalyst to form an at least partly reduced catalyst system which is free of nitrogen or has a reduced nitrogen content compared to the nitrogen-containing catalyst,
ii) at least partial regeneration of the at least partly reduced catalyst system to form an oxidic species which, if appropriate, has a reduced nitrogen content compared to the partly reduced catalyst system; suitable oxidic species already having been mentioned above;
iii) reaction of the oxidic species which, if appropriate, has a reduced nitrogen content compared to the partly reduced catalyst system with an amine component selected from ammonia, primary and secondary amines and ammonium salts;
it being possible for steps iii) and i) to be effected simultaneously or offset in time, or step iii) is effected first and then i).

19. The process according to claim 18, wherein steps i) and ii) are carried out successively, in which case steps i) and ii) are each passed through more than once.

20. The process according to claim 18, wherein the regeneration in step ii) is carried out in parallel to the reaction in step i).

21. The process according to claim 16, wherein the oxidic species is prepared in step a) of the process for the preparation of the nitrogen-containing catalyst by the following steps:
aa) precipitation of the desired metal compounds from a solution of their salts, for example of the nitrates, by addition of the base, for example ammonium carbonate, sodium hydroxide, ammonium hydroxide, lithium hydroxide, sodium carbonate, sodium hydrogencarbonate, potassium carbonate or mixtures thereof, to form the corresponding metal oxides or metal oxide hydroxides;
ab) filtering, washing and drying of the metal oxides or metal oxide hydroxides to obtain oxidic complexes;
ac) if appropriate calcination;
ad) if appropriate reduction of the resulting oxidic complexes with hydrogen; and
ae) if appropriate reoxidation with a defined amount of oxygen in order to obtain the desired oxidic species,
it being possible to carry out either step ac) or steps ad) and ae) or steps ac), ad) and ae).

22. The process according to claim 21, wherein Ni and Cu being present in at least two oxidation states, and the reoxidation in step ad) is effected with an amount of oxygen which is required to attain a molar metal/metal oxide ratio of from 0 to 500.

23. The process according to claim 16, wherein the reaction of the oxidic species in step b) of the process for the preparation of the nitrogen-containing catalyst with a gaseous amine component is effected at temperatures of from −35° C. to 600° C. and/or pressures of from 0.1 to 350 bar and/or for a period of from 0.001 to 10 hours.

24. The process according to claim 16, wherein the reaction of the oxidic species in step b) of the process for the preparation of the nitrogen-containing catalyst is effected with a liquid or solid amine component, by kneading the amine component into the oxidic species and subsequently heating to a temperature of from 50 to 600° C. for a period of from 0.1 to 20 hours.

25. A process for preparing nitrogen-containing catalysts, comprising:
a) preparation of an oxidic species comprising the following components:
nickel as metal M, it being possible for the nickel to be present in different oxidation states;
either copper alone or copper together with Mo and optionally W as promotor P¹ and at least one further promotor P³ selected from Rh and Ag, wherein the at least one further promotor P³ may—at least partly—be alloyed with nickel and/or copper; and a support material selected form ZrO₂ and magnesium—aluminum-oxide;
if appropriate one or more elements R selected from hydrogen, alkali metals and alkaline earth metals;
if appropriate one or more elements Q selected from chloride and sulfate;
oxygen, the molar proportion of oxygen being determined by the valency and frequency of the elements in the oxidic species other than other oxygen;
b) reaction of the oxidic species with an amine component selected from ammonia, primary and secondary amines and ammonium salts,
the nitrogen-containing catalyst being formed with the formation of water.

26. A process for the direct amination of hydrocarbons carried out in the presence of the oxidic species preparable according to claim 25.

27. A nitrogen-containing catalyst preparable by the process of claim 25.

28. The nitrogen-containing catalyst according to claim 27, consisting of:
from 10 to 80% by weight of nickel as metal M; and Cu as a promoter P¹, it being possible for M and Cu to be present at least partly in the form of the corresponding oxides:
from 0.1 to 10% by weight of molybdenum and/or tungsten as further promoter P¹;
from 5 to 60% by weight of Zr as a promoter P², Zr being present in the form of ZrO₂:
from 0.1 to 5% by weight of Rh or Ag as promoter P³;
from 0 to 15% by weight of one or more elements R selected from hydrogen, alkali metals and alkaline earth metals;
from 0 to 5% by weight of one or more elements Q selected from chloride and sulfate; and
oxygen, the molar proportion of oxygen being determined by the valency and frequency of the non-oxygen elements M, P¹, P², P³, R and Q;
where the sum total of the aforementioned components is 100% by weight; and
from 0.0001 to 20% by weight, based on the sum total of the aforementioned components, of nitrogen.

29. The process as claimed in claim 26, wherein the process is carried out in the presence of an oxidic species consisting of from 10 to 80% by weight of nickel and copper, from 0.1 to 10% by weight of molybdenum and/or tungsten, from 0.1 to 5% by weight, of Rh or Ag, From 5 to 60% by weight of Zr, Zr being present in the form of ZrO₂ and oxygen, the molar proportion of oxygen being determined by the valency and amount of the non-oxygen elements nickel, Cu, Mo, W or Ag and Zr, the sum total of the components in the oxidic species being 100% by weight; or wherein the oxidic species having, instead of from 5 to 60% by weight of Zr, Zr being present in the form of ZrOa from 5 to 60% by weight of Mg+Al, Mg+Al being present in the form of magnesium aluminum oxide, and instead of from 0.1 to 10% by weight of molybdenum and/or tungsten, from 0 to 10% by weight of molybdenum and/or tungsten.
